(19) United States
(12) Patent Application Publication
(10) Pub. No.: US 2008/0254439 A1
(43) Pub. Date: Oct. 16, 2008

(54) Plant Extract and Use Thereof as a Cryoprotective Agent

(76) Inventors: Fathey Sarhan, Montreal (CA); Francine Hamel, St-Hubert (CA); Melanie Grondin, Lasalle (CA); Diana Averill-Bates, Montreal (CA); Francine Denizeau, Montreal (CA); Jacques Denizeau, Montreal (CA)

Correspondence Address: GOUDREAU GAGE DUBUC, 2000 MCGILL COLLEGE, SUITE 2200, MONTREAL, QC, H3A 3H3, CA

(21) Appl. No.: 12/067,548

(22) PCT Filed: Sep. 22, 2006
(86) PCT No.: PCT/CA2006/001565

§ 371 Date: Mar. 20, 2008

Related U.S. Application Data
(60) Provisional application No. 60/719,188, filed on Sep. 22, 2005.

(51) Int. Cl. A01N 1/02; C12N 5/08

(52) U.S. Cl. 435/1.3; 435/374; 435/366; 435/370

(57) ABSTRACT

A cryopreservation medium comprising a protein-containing plant extract is disclosed. Methods, compositions, uses and kits for cryopreservation of biological material, such as a molecule, organelle, cell, embryo, tissue or organ, are also disclosed.

### CROSS REFERENCE TO RELATED APPLICATIONS

This application claims the benefit of U.S. Provisional Patent Application No. 60/719,188 filed Sep. 22, 2005, which is incorporated herein by reference in its entirety.

### FIELD OF THE INVENTION

The present invention relates to the cryopreservation of cells or tissues. More specifically, the present invention is concerned with the use of a plant-derived extract as a cryoprotective agent for such cryopreservation.

### BACKGROUND OF THE INVENTION

Freezing has been utilized for a number of years as an approach to preserve living cells. However, the cryopreservation and recovery of living cells has proven difficult, as the relatively harsh conditions of both the freezing and thawing of cells during their cryopreservation results in low viability of thawed cells. Various strategies have been pursued in order to improve the viability of thawed cells, relating mostly to the development of cryoprotective agents and improved methods of cryoprotection (e.g., improved rates of cooling). The industry standard cryoprotective agent for a variety of cell types is dimethyl sulfoxide (DMSO). However, despite its widespread use, DMSO can be toxic to cells and have adverse effects on cell function, which can for example compromise the use of such thawed cells for a variety of applications.

An example of a cell type which exhibits a variety of physiologically relevant functions is the hepatocyte. Among the liver cell types, hepatocytes are the most important for the function of the organ, representing about 70% of the total cellular population and 80% of hepatic tissue volume (1). They are responsible for the majority of hepatospecific functions (2) such as synthesis and secretion of essential proteins (e.g., ceruloplasmin, clotting factors, albumin). Hepatocytes are also involved in the biotransformation of endogenous and exogenous hydrophobic compounds (xenobiotics, toxicants) into water-soluble products that can be easily excreted into the extracellular medium (e.g., urine, bile) (3).

Hepatocytes thus represent a physiologically relevant model of the liver, especially as an in vitro experimental system for the evaluation of the metabolic fate and biological effects of xenobiotics. For xenobiotics that are mainly metabolized by the liver, the use of hepatocytes is more likely to yield results which are representative of those obtained in vivo, both in terms of metabolic profiles and rates of metabolic clearance (4-6).

Traditionally, freshly isolated hepatocytes are required for most studies on xenobiotic metabolism and toxicity, as for example, major xenobiotic-metabolizing enzymes such as the inducible isoforms of cytochrome P450 (CYP) decline rapidly in culture. However, this requires the use of freshly-procured livers for the preparation of hepatocytes for experimentation. Therefore, the cryopreservation of freshly isolated hepatocytes, retaining high viability and adequate liver functions after thawing, would significantly decrease the need for such fresh tissue. As such, cryopreserved hepatocytes of high quality would be of considerable value for investigations in the fields of hepatology, pharmacology and toxicology (7-9).

The major problems with the classical methods of cryopreservation of hepatocytes are the low survival rate in culture and poor metabolic activity and functional integrity. In addition, hepatocytes do not replicate in culture, as is the case for cell lines. Thus an efficient method of cryopreservation is necessary to reduce the cellular and functional damage incurred in hepatocytes during freezing. Several cryoprotective agents, such as DMSO noted above, are currently used to protect cells from dehydration caused by the formation of intracellular ice during freezing. However, they are either toxic to the cells and need to be eliminated rapidly after freezing (10) or cause osmotic stress that affects the metabolic competence of the cell (11). Consequently, the cryopreserved cell does not represent the native metabolic state of the cells or tissues and makes the interpretation of results obtained during the study of such cells erroneous.

There therefore exists a continued need for improved approaches for the cryoprotection of cells.

The present description refers to a number of documents, the content of which is herein incorporated by reference in their entirety.

### SUMMARY OF THE INVENTION

The invention relates to reagents and methods for cryopreservation based on the use of a plant extract.

More specifically, in accordance with the present invention, there is provided a cryopreservation medium comprising a protein-comprising plant extract.

The invention further provides a composition comprising the above-noted medium and a biological material. In an embodiment, the composition is frozen.

The invention further provides a method for cryopreserving a biological material, the method comprising freezing a suspension of the biological material in the above-noted medium.

The invention further provides a method for cryopreserving a biological material, the method comprising introducing the biological material into the above-noted medium and freezing the medium comprising the biological material.

The invention further provides a kit or package comprising the above-noted medium.

The invention further provides a use of the above-noted medium for cryopreservation of a biological material.

The invention further provides a protein-comprising plant extract for use in cryopreservation.

The invention further provides a composition comprising the above-mentioned extract and a biological material. In an embodiment, the composition is frozen.

The invention further provides a kit a package comprising the above-mentioned protein-comprising plant extract together with instructions for the cryopreservation of a biological material.

The invention further provides a use of the above-mentioned extract for cryopreservation of a biological material.

The invention further provides a method for cryopreserving a biological material, the method comprising introducing the above-mentioned extract into a cryopreservation medium prior to freezing.

In an embodiment, the above-noted extract is derived from a non-acclimated plant.

In an embodiment, the above-noted extract is derived from a cold-acclimated plant.

In an embodiment, the above-noted extract is derived from the aerial parts or leaf tissue of a plant.

In an embodiment, the above-noted plant is of a plant family selected from Poaceae (Gramineae), Leguminoseae, and Amaranthaceae.

In an embodiment, the above-noted plant is selected from wheat, rye, barley, alfalfa and spinach.

In an embodiment, the above-noted extract is substantially soluble.

In an embodiment, the above-noted extract is protein-enriched.

In an embodiment, the above-mentioned protein-enriched extract is prepared by salt precipitation. In a further embodiment, the above-mentioned salt precipitation is ammonium sulfate precipitation.

In an embodiment, the above-noted medium or extract is substantially free of DMSO.

In an embodiment, the above-noted medium or extract is substantially free of exogenous animal serum (e.g., fetal bovine serum). In a further embodiment, the above-noted medium is substantially free of both DMSO and exogenous animal serum.

In an embodiment, the above-mentioned medium or extract is substantially free of gluten.

In embodiments, the above-mentioned medium or extract is substantially free of (a): DMSO, (b): exogenous animal serum (e.g., fetal bovine serum), (c): gluten, (d): (a) and (b), (e): (a) and (c), (f): (b) and (c), or (g): (a), (b) and (c).

In an embodiment, the viability after thawing of the above-noted biological material cryopreserved in the above-noted medium is greater than or equal to 40%, in a further embodiment, greater than or equal to 50%, in yet a further embodiment, greater than or equal to 60%.

In an embodiment, the level or activity of a functional parameter after thawing of the above-noted biological material cryopreserved in the above-noted medium is greater than or equal to 40%, in a further embodiment, greater than or equal to 50%, in yet a further embodiment, greater than or equal to 60%. In embodiments, the functional parameter is selected from plating efficiency, adherence, cellular morphology, cellular secretion, protein synthesis, ammonium detoxification and enzyme activity.

In an embodiment, the above-noted medium or extract is for cryopreservation of a biological material selected from a molecule, organelle, cell, embryo, tissue and organ. In an embodiment, the cell is a eukaryotic cell. In an embodiment, the cell is a primary cell, a cell line or an immortalized cell. In an embodiment, the cell, embryo, tissue or organ is a mammalian cell, embryo, tissue, or organ. In an embodiment, the cell, embryo, tissue or organ is a human cell, embryo, tissue or organ. In an embodiment, the cell or tissue is a hepatocyte or hepatic tissue.

Other advantages and features of the present invention will become more apparent upon reading of the following non-restrictive description of specific embodiments thereof, given by way of example only with reference to the accompanying drawings.

### BRIEF DESCRIPTION OF THE DRAWINGS

FIG. 1: Cryopreservation potential of wheat protein extracts (WPEs) on isolated rat hepatocytes. Analysis of the viability of suspensions of rat hepatocytes after freezing was evaluated with the calcein/PI test by flow cytometry. Viability of rat hepatocytes (1.5×10⁶ cells/ml) was evaluated after 7 days of freezing in WME 10% FBS supplemented with 50% FBS (FBS), 20 mg of BSA (BSA) or 20 mg of E. coli proteins (E. coli), 15% DMSO and 20 mg of BSA (DMSO+BSA) or 15% DMSO and 50% FBS (DMSO). The effect of WPEs on viability of the suspension of rat hepatocytes was also evaluated after 7 days of freezing in WME supplemented with 20 mg of WPEs CA (cold-acclimated) or NA (non-acclimated). Freshly isolated hepatocytes (Fresh) served as a reference. Data (mean ±SEM) represent triplicate measurements of at least six independent experiments with different cell preparations (n=18).

FIG. 2: Viability of fresh and cryopreserved hepatocytes after seeding: effect of WPEs. Viability determination using LDH assay over a 4 day period after seeding thawed rat hepatocytes that had been cryopreserved for 7 days in WME supplemented with 15% DMSO and 50% FBS (DMSO), WPEs NA and CA. Viability (%) was obtained by subtracting the LDH released by dead or damaged hepatocytes from the total LDH in cells. Total LDH was evaluated by lysing cells with 10% Triton X-100. The release of LDH into the medium measured the loss of hepatocyte viability in culture, providing indirect measurement of the membrane integrity of cells. Freshly isolated hepatocytes served as a reference. Controls have been done to subtract intrinsic plant activity. Data (mean SEM) represent triplicate measurements from four experiments with different cell preparations (n=12). Statistical significance: * p<0.05, ** p<0.01 and *** p<0.001.

FIG. 3: Analysis of adherence and cellular morphology of cryopreserved rat hepatocytes by confocal microscopy. Adherence was visualized 24 h after seeding thawed rat hepatocytes, which had been cryopreserved for 7 days in WME 10% FBS supplemented with 15% DMSO and 50% FBS (B), WPEs NA (C) or CA (D). Freshly isolated hepatocytes, (A) served as a reference. Arrows indicate cell-to-cell contacts. Rat hepatocytes, 175×10³ cells, were visualized by confocal microscopy under 40× Hoffman (A-D). Photographs of cells are shown from a representative experiment, which was repeated at least in triplicate.

FIG. 4: Albumin secretion and detoxification of ammonium to urea by fresh and cryopreserved hepatocytes: beneficial effect of WPEs. (A) Albumin secretion (μg/10⁶ cells/24 h) in the cell culture medium over a 4 day period after seeding thawed rat hepatocytes and (B) production of urea (μg/10⁶ cells) during 24 hour time intervals after 1, 2 and 3 days in culture, for thawed rat hepatocytes that had been cryopreserved for 7 days in WME supplemented with 15% DMSO and 50% FBS (DMSO), WPEs NA and CA. Freshly isolated hepatocytes served as a reference. Controls have been done to subtract intrinsic plant activity. Data (mean ±SEM) represent triplicate measurements from four experiments with different cell preparations (n=12). Statistical significance: * p<0.05, ** p<0.01 and *** p<0.001.

FIG. 5: Activity and expression of the cytochrome P450 isoenzymes in fresh and cryopreserved hepatocytes: effect of WPEs. (A) Activity of the cytochrome P450 isoforms CYP1A1 and CYP2B and (B) expression of isoform CYP1A1, 48 h after seeding thawed rat hepatocytes that had been cryopreserved for 7 days in WME supplemented with 15% DMSO and 50% FBS (DMSO), WPEs NA and CA. Freshly isolated hepatocytes (Fresh) served as a reference. (A) The induction rate of the cytochrome P450 isoforms was measured by EROD (CYP1A1) and PROD (CYP2B) assays after a 24 h induction with benzo-a-pyrene. (B) Expression of the cytochrome P450 isoform CYP1A1 after a 24 h induction with benzo-a-pyrene (+). Immunodetection with CYP1A1 antibody on 30 μg of mammalian protein extracts after a 24 h induction with benzo-a-pyrene (+) and quantification by densitometry. The rate of induction is the ratio between the density of the non-induced on the density of the induced lane. Freshly isolated hepatocytes (Fresh) and Fresh+NA were also tested. Membrane staining was used as protein loading charge control. Freshly isolated hepatocytes (Fresh) served as a reference. Data (mean ±SEM) represent triplicate measurements from four experiments with different cell preparations (n=12). Immunodetection (B) of proteins is shown from one representative experiment, which was repeated at least in triplicate. Statistical significance: p<0.05.

FIG. 6: Cryopreservation potential of the PEs on isolated rat hepatocytes. Analysis of the viability of suspensions of rat hepatocytes after freezing was evaluated with the calcein/PI test by flow cytometry. Viability of rat hepatocytes (1.5×10⁶ cells/ml) was evaluated after 7 days of freezing in WME 10% FBS supplemented with 15% DMSO and 50% FBS (DMSO). The effect of PEs from wheat (Triticum aestivum) cv Clair, wheat cv Glenlae, barley (Hordeum vulgare), rye (Secale cereale), alfalfa (Medicago sativa) or spinach (Spinacia oleracea) on viability of the suspension of rat hepatocytes was also evaluated after 7 days of freezing in WME supplemented with 20 mg or 40 mg (+) of NA plant PEs. Freshly isolated hepatocytes (Fresh) served as a reference. Data (mean ±SEM) represent duplicate measurements of at least three independent experiments with different cell preparations (n=6).

FIG. 7: Cryopreservation of eukaryotic cells with DMSO and WPEs. Analysis of the viability of suspensions of eukaryotic cells after freezing was evaluated with calcein/PI test by flow cytometry. Viability of primary rat hepatocytes cells, A549 (human lung carcinoma), Caco-2 (human colorectal adenocarcinoma), CHO-BL (Chinese hamster ovary transfected with TGF-b1 cDNA), HeLa (cervical cancer cells taken from Henrietta Lacks), HIEC (human intestinal epithelium cell) and Jurkat (Human T cell leukemia) cell lines (1.5×10⁶ cells/ml) was evaluated after 7 days of freezing in their respective growth media supplemented with 15% DMSO and 50% FBS (DMSO) or WPEs NA Clair (NA). Freshly isolated hepatocytes (Fresh) served as a reference. Data (mean ±SEM) represent triplicate measurements of at least three independent experiments with different cell preparations (n=9).

FIG. 8: Cryopreservation potential of the WPEs proteins from ammonium sulfate precipitate fractions on isolated rat hepatocytes. Viability of suspensions of hepatocytes (1.5×10⁶ cells/ml) after 7 days of freezing was evaluated with calcein/PI by flow cytometry. Hepatocytes were frozen in WME 10% FBS (WME), supplemented with 15% DMSO and 50% FBS (DMSO) or 20 mg of NA (non-acclimated) or CA (cold-acclimated) WPE (WPE) or 20 mg of NA or CA protein fraction 41-60% (41-60) or 20 mg of NA or CA protein fraction 61-80% (61-80) or 20 mg of NA or CA protein fraction 81-100% (81-100). Freshly isolated hepatocytes (Fresh) served as reference. Data (mean ±SEM) represent triplicate measurements of three different preparations of WPEs in three independent experiments with different cell preparations (n=27).

FIG. 9: Influence of the fetal bovine serum (FBS) on the cryopreservation potential of the NA (non-acclimated) WPE on isolated rat hepatocytes. Viability of suspensions of hepatocytes (1.5×10⁶ cells/ml) after 7 days of freezing was evaluated with calcein/PI by flow cytometry. Hepatocytes were frozen in WME, supplemented with 15% DMSO and 50% FBS (DMSO) or 15% DMSO (DMSO-FBS) or 20 mg of NA WPE and 10% FBS (NA) or 20 mg of NA WPE (NA-FBS). Freshly isolated hepatocytes (Fresh) served as reference. Data (mean ±SEM) represent triplicate measurements of two different preparations of WPEs in three independent experiments with different cell preparations (n=18).

FIG. 10: Gluten quantification of the NA (non-acclimated) and CA (cold-acclimated) WPEs. Quantitative analysis of the gluten content was evaluated by a sandwich enzyme immunoassay. Data (mean ±SEM) represent duplicate measurements of two different preparations of WPEs (n=4).

### DESCRIPTION OF ILLUSTRATIVE EMBODIMENTS

In the studies described herein, Applicants have developed an improved method of cryopreservation for cells, such as isolated hepatocytes, conducive to long term storage by freezing, such as storage in liquid nitrogen. Applicants surprisingly found that when cells, including hepatocytes as well as various cell lines, were cryopreserved with plant (e.g. wheat) extracts, cellular viability after thawing was equivalent to or better than that of cells that were cryopreserved with DMSO. When hepatocytes that had been cryopreserved with plant extracts were thawed and seeded in culture dishes, their morphology was similar to that of fresh cells. Furthermore, hepatospecific functions such as albumin secretion and biotransformation of ammonium to urea were well maintained during 4 days in culture. Hepatospecific functions were comparable to those of fresh cells, which was in contrast to hepatocytes that had been cryopreserved with dimethyl sulfoxide. The levels of induction of cytochrome P450 isoenzymes CYP1A1 and CYP2B in hepatocytes that had been cryopreserved with wheat extracts were similar to those in fresh hepatocytes.

These findings clearly show that plant extracts, such as wheat extracts, are a much better cryopreservant for cells, (e.g., primary cells such as primary hepatocytes), than dimethyl sulfoxide. Such extracts provide the further advantages of being a natural product and represent an efficient, non-toxic, economic and user-friendly cryopreservant with wide applications to different biological systems. This cryopreservation method permits long-term storage and the recovery of large quantities of healthy cells, which maintain their differentiated functions, such as in the case of hepatocytes.

Accordingly, in a first aspect, the invention provides a protein-comprising plant extract for use in cryopreservation. “Protein-comprising plant extract” as used herein refers to an extract or preparation obtained from plant material in such a way that it comprises protein from the plant material. In an embodiment, such an extract may be a crude extract, obtained for example from the grinding (e.g., in a blender or similar device) of plant material in a suitable solvent (e.g., an aqueous solvent [e.g. water]), which may be followed by suitable means to remove particulate matter (e.g., filtration, centrifugation).

In a further aspect, the invention provides a cryopreservation medium comprising a protein-comprising plant extract.

The medium or extract of the invention may in embodiments be provided in a ready to use form, a concentrated form (i.e., requiring dilution), or in a dehydrated form (i.e., requiring reconstitution with a suitable aqueous solvent (e.g., water)). Such forms of the medium of the invention may in embodiments be provided in suitable kits or packages, in further embodiments together with instructions (e.g., written and/or graphic material and/or on a computer-readable form) for their use, preparation and/or reconstitution. The invention further provides kits or packages containing such forms of media or extract, in further embodiments together with instructions for its reconstitution/rehydration, dilution or generally their preparation.

In a further aspect, the invention provides a composition comprising the above-mentioned extract and a biologically-compatible or -acceptable carrier or vehicle.

In a further aspect, the invention provides a composition comprising the above-mentioned medium and a biological material.

In a further aspect, the invention provides a method of preparing the above-mentioned medium, comprising introducing a protein-comprising plant extract into a solution suitable for storage or culturing of a biological material.

In a further aspect, the invention provides a method of preparing the above-mentioned composition, comprising introducing a biological material into the above-mentioned medium.

In a further aspect, the invention provides a method for cryopreserving a biological material, comprising freezing a suspension or mixture of the biological material in the above-mentioned medium.

In a further aspect, the invention provides a method for cryopreserving a biological material, comprising introducing or suspending the biological material into the above-mentioned medium and freezing the suspension or mixture of the biological material in the above-mentioned medium.

In a further aspect, the invention provides a use of the above-mentioned medium or extract for the cryopreservation of biological material.

In a further aspect, the invention provides a method for cryopreserving a biological material, said method comprising introducing the above-mentioned extract into a cryopreservation medium prior to freezing.

In a further aspect, the invention provides a kit or package comprising the above-mentioned medium or extract. In an embodiment, the kit or package may further comprise instructions (e.g., written and/or graphic material) for the cryopreservation of biological material.

In a further aspect, the invention provides a package comprising the above-mentioned composition. In an embodiment, the composition is frozen, in which case the package may further comprise instructions (e.g., written and/or graphic material) for thawing the composition.

The medium, extract and methods of the invention are advantageous in that cryopreservation may be performed in the absence of traditional chemical cryoprotectants such as DMSO.

The medium, extract and methods of the invention are also advantageous in that cryopreservation may be performed in the absence of components obtained from animal sources, such as exogenously added animal serum (e.g. fetal bovine serum, fetal calf serum), thus reducing the risk of contamination by pathogens transmitted from such animal sources during the preparation of such components. In an embodiment, the medium and extract of the invention are substantially free of both chemical cryoprotectants (e.g. DMSO) and exogenous animal serum (e.g. fetal bovine serum, fetal calf serum).

In a further embodiment, the medium and extract of the invention are substantially gluten-free (or substantially free of gluten). “Substantially gluten-free” as used herein refers to a gluten level of 200 ppm or less in the medium or extract.

In further embodiments, the medium and extract of the invention are substantially free of DMSO, exogenous animal serum (e.g., fetal bovine serum), gluten, or any combinations thereof.

“Medium” or “media” as used herein refers to a solution which is conducive to supporting biological material, such as cells, in a viable state. Such media typically contain for example suitable means to maintain isotonicity and buffering means for maintaining pH in accordance with the biological material of interest. Such media may also contain other additives which are known in the art, such as agents to maintain or promote cell growth, agents to inhibit microbial growth (e.g., an antibiotic), and/or a pH indicator agent.

In an embodiment, the above-mentioned plant extract is derived from a non-acclimated plant or tissue thereof. “Non-acclimated” as used herein refers to a plant which is not expressing freezing-tolerance or anti-freeze proteins. This term thus encompasses (a) plants which do not comprise genes encoding freezing-tolerance or anti-freeze proteins, (b) plants which cannot undergo induction of expression of freezing-tolerance or anti-freeze proteins, and (c) plants which are capable of induction of expression of freezing-tolerance or anti-freeze proteins, but are not subjected to such induction when the plant material is obtained to prepare the extract. In the latter case, such an absence of induction typically means that the plant has not been exposed to cold acclimation conditions (temperature lower than 15° C.) for such induction to occur. In contrast, a “cold-acclimated” plant as used herein refers to a plant which is not only capable of undergoing induction of expression of cold regulated or freezing-tolerance associated or anti-freeze proteins but has been so induced by suitable treatment (e.g. cold treatment) and is therefore expressing such proteins. Thus, a plant capable of acclimation (e.g. winter wheat) would be a “non-acclimated” plant in the absence of such induction, and would be considered a “cold-acclimated” plant if it has been so induced.

In an embodiment, the above-mentioned plant extract is derived from a cold-acclimated plant or tissue thereof. “Cold-acclimated” as used herein is defined above.

In an embodiment, the above-mentioned plant extract is obtained from a plant tissue other than seed tissue. In an embodiment, the above-mentioned plant extract is obtained from the aerial parts or leaf tissue of a plant.

In embodiments, the plant is of a plant family selected from Poaceae (Gramineae), Leguminoseae, and Amaranthaceae. In a further embodiment, the plant is selected from wheat (e.g., wheat (Triticum aestivum) cv Clair, wheat cv Glenlae), barley (e.g., Hordeum vulgare), rye (e.g., Secale cereale), alfalfa (e.g., Medicago sativa) or spinach (e.g., Spinacia oleracea).

In an embodiment, the above-mentioned plant extract is substantially soluble. “Substantially soluble” as used herein refers to a solution in which virtually all solute is dissolved in the solvent and appears to be clear to the naked eye. Substantially soluble solutions may be prepared by a number of methods known in the art, including mechanical methods of removing particulate, undissolved matter (e.g., filtration, centrifugation) or by adding or removing components or treating the solution to enhance solubility.

In an embodiment, the above-mentioned plant extract is protein-enriched. “Protein-enriched” as used herein refers to a preparation which has undergone a treatment which results in a greater concentration of protein in the preparation following such treatment, or results in a greater amount of protein relative to other components of the preparation, following such treatment. This term thus also encompasses a preparation which has undergone a treatment to retain, separate, isolate or purify proteins to a greater extent than one or more of the other components present in the preparation prior to such treatment. Suitable treatments are known in the art, and include for example ultrafiltration/microfiltration, centrifugation, chromatography, electrophoresis and precipitation (e.g., ammonium sulfate precipitation). In an embodiment, the protein-enriched plant extract is obtained by ammonium sulfate precipitation. In an embodiment, the protein-enriched plant extract is the precipitate fraction obtained at greater than 40% ammonium sulfate (e.g. the 41-100% fraction), in a further embodiment, the 41-80% ammonium sulfate fraction, in a further embodiment, the 41-60% ammonium sulfate fraction. In a further embodiment, the protein-enriched plant extract is the precipitate fraction obtained at greater than 60% ammonium sulfate (e.g. the 61-100% fraction), in a further embodiment, the 61-80% ammonium sulfate fraction. In a further embodiment, the protein-enriched plant extract is the precipitate fraction obtained at greater than 80% ammonium sulfate, e.g., the 81-100% ammonium sulfate fraction.

As noted above, an advantage of the medium of the invention is that it may allow cryopreservation in the absence of DMSO. Accordingly, in an embodiment, the above-mentioned medium is “substantially free of DMSO”, which, as used herein, refers to a medium to which DMSO has not been directly added as a cryoprotectant.

As noted above, another advantage of the medium of the present invention is that it may allow cryopreservation in the absence of component from animal sources such as animal serum. Accordingly, in an embodiment, the above-mentioned medium is substantially free of exogenous animal serum (i.e., in cases where biological material from an animal source is pering cryopreserved, exogenous serum represents serum derived from a different animal than the source of the biological material). In a further embodiment, the above-mentioned medium is substantially free of fetal bovine serum. As used herein, “substantially free of animal serum” or “substantially free of fetal bovine serum” refer to a medium to which animal serum or fetal bovine serum has not been directly added. In an embodiment, the above-mentioned medium is substantially free of both DMSO and exogenous animal serum (e.g. fetal bovine serum).

“Biological material” as used herein refers to any material derived from a biological system, including but not limited to material containing genetic information and which is capable of self-reproduction or reproduction in a suitable biological system. In embodiments, the biological material is selected from a molecule, organelle, cell, embryo, tissue or organ. In embodiments, the biological material is eukaryotic or prokaryotic. In an embodiment, the cell is a primary cell. “Primary cell” as used herein refers to a cell obtained directly from a living organism, which is not immortalized. In embodiments, the cell is eukaryotic. In an embodiment, the cell, embryo, tissue or organ is an animal cell, embryo, tissue or organ, in a further embodiment, a mammalian cell, embryo, tissue or organ, in yet a further embodiment, a human cell, embryo, tissue or organ. In a further embodiment, the cell is a hepatocyte, such as a primary human hepatocyte. In further embodiments, the material is a cell line or immortalized cell.

In an embodiment, the viability of the biological material (e.g., cell, tissue or organ) following cryopreservation with the above-mentioned medium, i.e., determined following thawing, will be at least 40% of the initial viability (i.e., the viability of the biological material prior to cryopreservation). In further embodiments, the viability will be at least 45%, 50%, 55%, 60%, 65%, 70% or 75% of the initial viability. Methods to determine viability are known in the art. For example, certain suitable methods to determine viability are described in the Examples below.

In an embodiment, the level or activity of a functional parameter (e.g., which reflects metabolic activity) of the biological material (e.g., molecule, organelle, cell, embryo, tissue or organ) following cryopreservation with the above-mentioned medium, i.e., determined following thawing, will be at least 40% of the initial level or activity of the functional parameter (i.e., the level or activity prior to cryopreservation). In further embodiments, the level or activity of a functional parameter will be at least 45%, 50%, 55%, 60%, 65%, 70% or 75% of the initial level or activity of the functional parameter. In embodiments, the functional parameter is selected from plating efficiency, adherence, cellular morphology, secretion (e.g. of albumin), protein synthesis, ammonium detoxification and enzyme (e.g. LDH, different isoforms of cytochrome P450) activity. Methods to determine a level or activity of various functional parameters are known in the art. For example, certain suitable methods are described in the Examples below.

A composition (comprising the above-mentioned medium and biological material) of the invention may be prepared for freezing in a number of ways, in that the various components may be combined in different sequences. For example, in an embodiment, the biological material may be introduced into medium already comprising the plant extract. Another possibility may be for example to introduce the biological material into a medium solution and then subsequently introducing the plant extract to this mixture.

In embodiments, controlled freezing of the above-mentioned composition may be performed using a programmable freezing device, which facilitates reproducible and optimal cooling rates. Such devices are known in the art and are commercially available.

Preferred containers for freezing the above-mentioned composition are those that are stable at cryogenic temperatures and allow appropriate heat transfer for both freezing and thawing. Such containers include, for example, sealed plastic vials for small volumes (e.g., 1-2 ml) and polyolefin bags (typically held between metal plates for freezing) for larger volumes, both of which are known in the art and are commercially available.

Freezing is generally performed using suitable means (such as a freezer, the above-noted device, or contacting the biological material with an appropriate sub 0° C. substrate/bath) to lower the temperature of the sample to an appropriate temperature (e.g. −20° C.; −80° C.) at an appropriate rate. In embodiments, the frozen samples may be transferred to a vessel suitable for long-term cryogenic storage, such as those employing storage in liquid nitrogen (about −196° C.) or in liquid nitrogen vapor (about −105° C.). Such devices are known in the art and are commercially available.

The present invention is illustrated in further details by the following non-limiting examples.

### EXAMPLES

### Example 1

### Materials and Methods

Chemicals. Collagenase, insulin, Williams' medium E (WME), dimethyl sulfoxide (DMSO), resofurin and all other chemicals were obtained from Sigma Chemical Company (St-Louis, Mo.). Leibovitz medium (L-15), gentamicin and MEM vitamins were obtained from Gibco/Life Technologies (Burlington, ONT). Calcein, 7-ethoxyresorufin-O-deethylase (EROD) and 7-pentoxyresorufin-O-depentylase (PROD) were obtained from Molecular Probes (Eugene, Oreg.). Propidium iodide (PI) was obtained from Calbiochem (San Diego, Calif.). Antibodies for cytochrome P-450 μl (CYP1A1 (G-18) goat polyclonal IgG) and for anti-goat IgG (horse radish peroxidase (HRP) conjugated mouse anti-goat IgG) were obtained from Santa Cruz Biotechnology (Santa Cruz, Calif.). Fetal bovine serum (FBS) was obtained from Medicorp (Montreal, QC).

Plant materials and growth conditions. Winter wheat genotype (Triticum aestivum L. cv Clair, LT₅₀ (lethal temperature that kills 50% of the seedlings)−19° C. was used in this study. Wheat plants were grown and treated as previously described (12). Briefly, control plants were grown for 10 days at 20° C. and cold acclimation (CA) was performed at 4° C. for a 7-day period.

Total protein extraction. The aerial parts of the seedlings were collected and blended until a homogeneous solution was obtained with cold nanopure water. The homogenate was filtered through 3 layers of miracloth and centrifuged at 30 000 g for 45 min at 4° C. The pH of the supernatant was adjusted to 7.4 and sterilized using a 0.22 μm filter. The extract was concentrated by freeze-drying and stored at −20° C.

Hepatocyte isolation and culture. Hepatocytes were isolated from male Sprague-Dawley rats (120-180 g), obtained from Charles River Canada (Saint-Constant, QC), in a two-step collagenase digestion technique (13; 14). Animals were maintained and handled in accordance with the Canadian Council on Animal Care guidelines for the care and use of experimental animals (15). Cell viability was evaluated by flow cytometry (FACScan™, Becton Dickinson, Oakville, ON) with 2 μM PI (16). Isolated cells were diluted to 3.5×10⁵/ml and cultured in tissue culture plates (Corning, Acton, Mass.) in WME medium supplemented with 10% FBS, insulin (0.2 μg/ml) and gentamicin (50 μg/ml) in a humidified atmosphere of 5% CO₂ and 95% air at 37° C. After 3 h, the medium was changed and cells were incubated overnight in L-15 medium (16) supplemented with insulin and gentamicin.

Cryopreservation of isolated hepatocytes. Immediately after isolation, the hepatocyte suspension was added to a mixture of ice-cold WME medium supplemented with 10% FBS and non-acclimated (NA) or CA wheat protein extracts (WPEs) in cold cryovials. Positive (15% DMSO+50% FBS) and negative (WME) controls were also prepared. The tubes containing cells were then frozen at a cooling rate of 1° C./min in a controlled freezing container (Nalgene, Rochester, N.Y.) to −80° C. for one day, and then transferred to liquid nitrogen.

Thawing of cryopreserved hepatocytes. The frozen hepatocytes were thawed quickly by gentle agitation in a 37° C. water bath. Viability assays were performed on the hepatocyte suspension. For adherence and metabolic assays, the hepatocyte suspension was diluted 10-fold by addition of cold WME medium, immediately after thawing. A 30% isotonic Percoll centrifugation step was performed to remove dead cells when viability was lower than 80%. After centrifugation (4° C., 50 g, 2 min), hepatocytes were suspended in 10 ml of WME medium. Hepatocytes were washed twice as above, then suspended at 3.5×10⁵/ml in WME and cultured in tissue culture plates in WME medium supplemented with insulin and gentamicin in a humidified atmosphere of 5% CO₂ and 95% air at 37° C. After 3 h, the medium was changed and cells were incubated overnight in L-15 medium supplemented with insulin and gentamicin.

Viability assays. After freeze/thaw cycles, hepatocyte suspensions were stained with the fluorescent probes 4 μM calcein and 2 μM PI in WME medium for 5 minutes. The samples were analyzed by flow cytometry (excitation at 488 nm) using a Becton Dickinson FACScan™. The number of live cells expressing green fluorescence of calcein and the number of dead cells expressing red fluorescence of PI were determined with Cell Quest™ software (Becton Dickinson, Oakville, ON).

Lactate dehydrogenase (LDH) activity was determined in the medium of seeded hepatocytes as a measure of hepatocyte deterioration as described by Moldeus et al. (17). The hepatocyte culture medium was removed daily and the activity of the LDH released into the medium was quantified (18).

Plating efficiency. Plating efficiency was determined by measuring the LDH activity in cells prior to seeding and in 3 and 24 h-old cultures. Plating efficiency was defined as the LDH activity in 24 h-old cultures divided by the LDH activity in pre-culture cells.

Adherence and cellular morphology. Adherence and cellular morphology were evaluated by confocal microscopy. For the confocal microscopy observations, tissue culture plates coated with collagen were used. All analyses were carried out using the confocal microscope MRC1024 from BioRad (Microscience, Cambridge, Mass.) equipped with an argon laser (excitation at 488 nm) combined with an inverted microscope Eclipse Model TE 3000 (Nikon, Montreal, QC) with objectives of 40× Hoffman.

Determination of albumin secretion. Albumin secretion from hepatocytes was quantified every 24 h, until 96 h, in different hepatocyte culture media by the sandwich enzyme-linked immunosorbent assay (ELISA) according to Uotila et al. (19), with minor modifications (20). Briefly, 96-well plates (Nunc, Napierville, Ill.) were coated with anti-rat albumin rabbit antiserum (1 μg/ml). The plates were incubated for 30 min at room temperature (RT), then at 4° C. overnight, washed with phosphate buffered saline (PBS), blocked for 30 min at RT with 5% FBS in PBS, and then rewashed with PBS. Serial dilutions of sample and albumin standard were added (200 μl/well). The plates were incubated for III at RT and washed with PBS supplemented with 0.05% Tween-20 (PBS-T). Thereafter, the secondary antibody (peroxidase-conjugated anti-rat albumin; 2 μg/ml) was added and the plates were incubated for 1 h at RT. After 3 washings with PBS-T, the plates were incubated with the substrate o-phenylendiamine (OPND) (0.1 M NaH₂PO₄, 1 mg/ml OPND, 0.4 μl/ml H₂O₂) for 30 min at RT in the dark. The reaction was stopped with 4 M H₂SO₄. Albumin concentrations were determined at 550 nm using a standard curve ranging from 0 to 250 ng/dl of rat albumin using an ELISA reader (SPECTRAFluor Plus™, Tecan, Calif.).

Urea determination. To evaluate the hepatocyte-mediated biotransformation of ammonia to urea, seeded hepatocytes were exposed to L-15 culture medium containing 10 mM NH₄Cl. Samples of media were collected at the beginning and after 24 h intervals of exposure to ammonia, during 3 days. Urea concentration in the samples was measured calorimetrically using the urea nitrogen reagent set (BioTron Diagnostics, Hemet, Calif.) and an ELISA reader at 540 nm. Concentrations of urea were determined using a standard curve ranging from 0 to 45 mg/dl of urea. The results are presented as μg urea/10⁶ cells.

Enzymatic activity and protein expression of cytochrome P450 isoforms. CYP1A1 and 2B enzymatic activities were measured in hepatocyte cultures induced with benzo-a-pyrene (10 μM). Cells were washed 2 times with PBS and the substrates EROD (8 μM) or PROD (17 μM) (λ_{exc}: 530 nm; λₑₘ: 585 nm) were added to the culture dishes and incubated for 1 h. The supernatant (300 μl) was mixed with 200 μl of ETOH and the activity in 200 μl of the mixture was measured using an ELISA reader at 585 nm. Enzymatic activity was determined using a standard curve ranging from 0 to 200 μM of resorufin.

CYP1A1 protein expression was determined after a 24 h induction with benzo-a-pyrene (10 μM). Cells were washed with PBS and scraped off the plates, suspended in 100 μl of lysis buffer (20 mM Tris-HCl, 2 mM EGTA, 2 mM EDTA, 6 mM β-mercaptoethanol) and homogenized by sonication. Protein samples (30 μg) were mixed with Laemmli sample buffer and separated on a 12% SDS polyacrylamide gel (SDS-PAGE) (21). Electrophoresis was performed at 140 volts for 50 min. Transfer of proteins to a polyvinylidene fluoride (PVDF) membrane was performed at 80 mA/membrane for 1.5 h. Membranes were blocked with 5% dry milk in TBS-T buffer (2 mM Tris-HCl, 13.7 mM NaCl, 0.1% Tween) for 1 h at RT. Membranes were washed 3 times with TBS-T, incubated with a primary antibody (CYP1A1 (G-18), dilution 1/1000) overnight at 4° C., washed 3 times with TBS-T and then incubated for 1 h with the HRP-conjugated secondary antibody (anti-goat IgG, dilution 1/1000). The protein bands reacting with the antibody were revealed using western lightning chemiluminescence reagent plus™ (PerkinElmer Life Sciences, Boston, Mass.) and BioMax MS™ film (Eastman-Kodak, Rochester, N.Y.). Proteins on the film were quantified by densitometry using a Molecular Dynamics scanner (Amersham, Baie d'Urfe, Qc) and IP Lab gel software (Scanalytics Inc., Fairfax, Va.).

Statistical analysis. Quantitative results were expressed as mean ±SD of at least 3 replicate dishes for each condition with a minimum of 3 experimental repeats, with each experimental repeat using cells from a different hepatocyte isolation procedure. Data were normalized to non-cryopreserved experimental controls at each time interval in the same experiment. The comparison between groups and the analysis for differences between the means of the control and treated groups were performed using ANOVA followed by the post-hoc test Newman-Keuls (P<0.05). The threshold for statistical significance was considered p<0.05 (*), p<0.01 (**) and p<0.001 (***).

### Example 2

### Cryopreservation of Rat Hepatocytes Using Classical Techniques

Initial experiments were designed to determine the optimal cryopreservation protocol for freshly isolated rat hepatocytes using DMSO. Hepatocyte concentrations ranging from 1.5 to 5×10⁶ cells/ml were frozen to −80° C. in Williams' medium supplemented with 10% FBS and 5 to 25% DMSO. The best results were obtained when cells were cryopreserved with 15% DMSO. The rate of freezing was also assessed using three different freezing apparatus: styrofoam (4 h at −20° C., 18 h at −80° C.), a programmable freezer (−6° C./h until −20° C., then 18 h at −80° C.) or a Nalgene™ apparatus (−1° C./min until −80° C. for 18 h). The results indicated that a hepatocyte concentration of 5×10⁶ cells/ml and freezing in the Nalgene apparatus constituted the optimal conditions for the cryopreservation of rat hepatocytes using DMSO. These conditions were used as a reference for classical cryopreservation in the subsequent experiments.

### Example 3

### Cryopreservation Potential of WPEs on Rat Hepatocytes

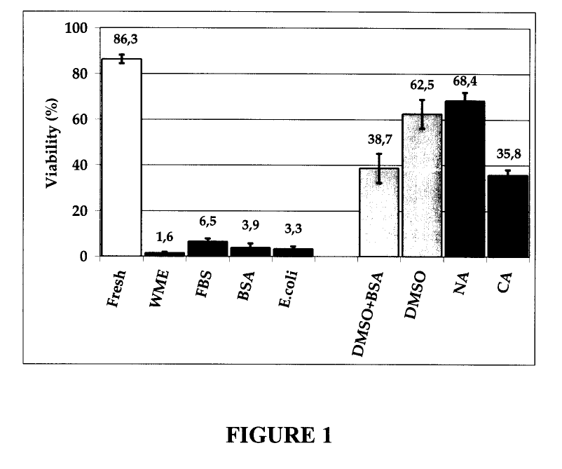

The ability of different WPEs to improve the viability of cryopreserved rat hepatocytes, compared to DMSO, was evaluated. Results in FIG. 1 present the viability of rat hepatocytes after 7 days of freezing in the presence of WPEs, other proteins and DMSO compared to fresh hepatocytes. The viability of hepatocytes with 15% DMSO+50% FBS (positive control) was 62.5%, compared to 86.3% for freshly isolated hepatocytes. When 15% DMSO+20 mg of BSA were used, viability of cryopreserved hepatocytes was 38.7%. On the other hand, low viability was obtained in the presence of 20 mg of BSA, WME medium, 20 mg of FBS or E. coli proteins (3.9, 1.6, 6.5 and 3.3%, respectively). However, significant results were obtained with the addition of 20 mg of NA WPE, giving viability of 68.4%. These levels of viability were comparable to that obtained with the classical cryoprotectant, DMSO. In comparison, an equal amount of CA WPE gave viability of 35.8% (FIG. 1). These findings demonstrate that the WPEs contain specific compounds with cryoprotective activity at least equivalent to the commonly used cryoprotectant, DMSO.

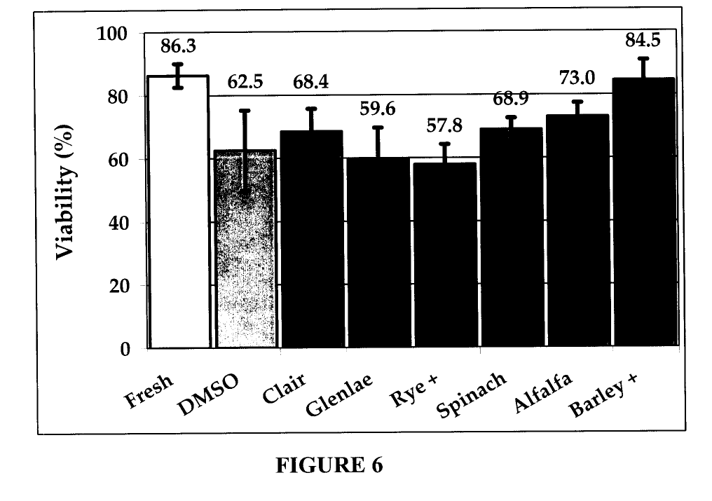

Our results also demonstrate that extracts from other plants such as barley, rye, alfalfa, and Spinach possessed cryoprotective activity for hepatocytes (see FIG. 6). An equal amount of other proteins such as BSA or E. coli proteins did not show any cryopreservation activity indicating that the cryoprotective activity is specific to plant extracts.

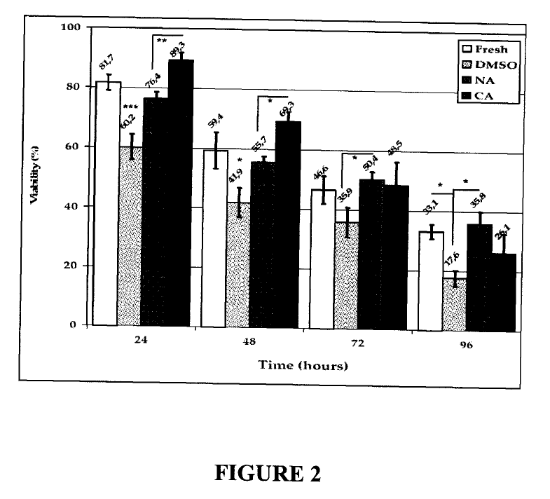

The viability of hepatocytes was also assessed using the release of LDH. The release of cellular LDH measures the loss of hepatocyte viability in culture by providing indirect measurement of the membrane integrity of cells. The results in FIG. 2 show that the levels of viability for hepatocytes cryopreserved with WPEs were better than that obtained with DMSO. After 24 h in culture, high viabilities of 76.4 and 89.3% were obtained for hepatocytes cryopreserved with the NA and CA WPEs, respectively, compared to 60.2% for the classical DMSO. Improved viability in the presence of WPEs, compared to DMSO, was maintained throughout the 96 h culture period. WPEs improved viability to similar levels as those obtained in fresh hepatocytes during 96 h. The LDH test performed on post-thaw hepatocytes following seeding further demonstrates that hepatocytes cryopreserved with WPEs maintained better cellular viability in culture than those that were cryopreserved with DMSO. It was further demonstrated that the cellular viability of the WPE cryopreserved hepatocytes was similar to that of fresh hepatocytes, indicating that WPEs are less toxic and more efficient as cryopreservation agents than DMSO.

### Example 4

### Plating Efficiency, Adherence and Cellular Morphology of the Cryopreserved Rat Hepatocytes

The ability of thawed cells to survive in culture is an indication of the successful cryopreservation of hepatocytes. The plating efficiency of cells was assessed 3 h and 24 h after seeding and culture. After 3 h in culture, the plating efficiencies of the thawed rat hepatocytes cryopreserved with DMSO, NA and CA WPEs were slightly lower than for the freshly isolated rat hepatocytes (62.5 to 64.9% compared to 77.3%, Table 1). After 24 h in culture, the plating efficiency was higher than 50% for the thawed rat hepatocytes cryopreserved with DMSO, NA and CA WPEs, relative to the non-cryopreserved hepatocytes (100%) (Table 1). These findings demonstrate that the hepatocyte plating efficiency was comparable in the presence of WPEs and the classical cryoprotectant DMSO.

TABLE 1
Plating efficiency of thawed rat hepatocytes following
cryopreservation with WPEs compared to freshly isolated hepatocytes

| Culture | | | | |
|---|---|---|---|---|
| period | | Cryopreserved | | |
| (hours) | Fresh | DMSO | NA | CA |
| 3 | 77.3 ± 0.5 | 64.9 ± 1.7 | 63.7 ± 1.5 | 62.5 ± 0.4 |
| 24 | 100.0 ± 2.4 | 50.3 ± 4.1 | 50.9 ± 2.9 | 52.5 ± 3.4 |

*Plating efficiency was evaluated by LDH activity in freshly isolated and cryopreserved hepatocytes, as described in the Materials and Methods. Data are expressed as a mean ± SEM from three different experiments.

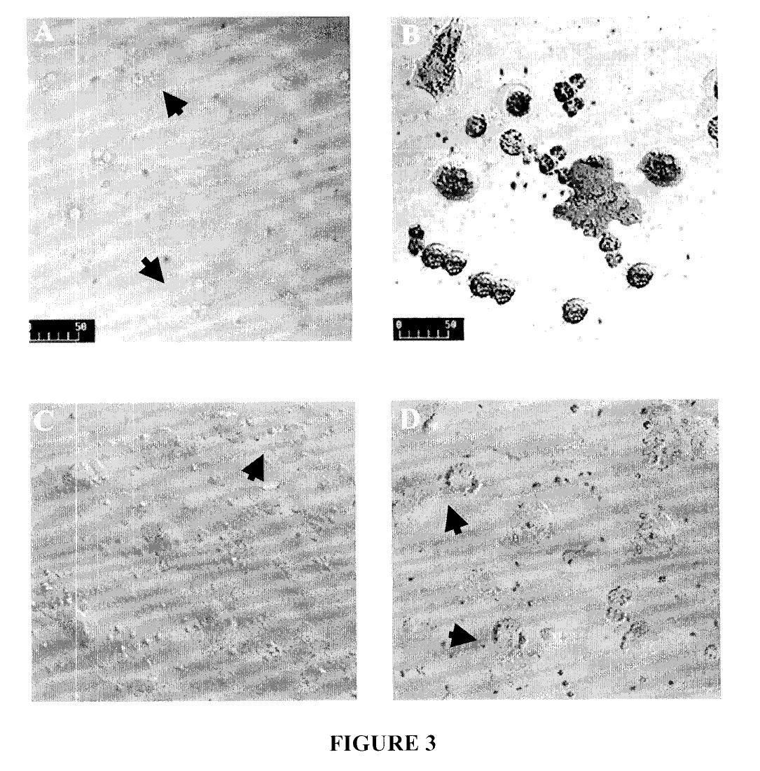

Morphological analysis of thawed cryopreserved hepatocytes by confocal microscopy is shown 24 h after seeding for hepatocytes that were cryopreserved with WPEs (FIGS. 3C and D) as well as with DMSO (FIG. 3B), compared to freshly isolated hepatocytes (FIG. 3A). At the same cell concentration, the fresh and WPE-cryopreserved hepatocytes showed similar cellular morphology. Hepatocytes cryopreserved with WPE (FIGS. 3C and D) appeared as the fresh cell (FIG. 3A) with a round cellular morphology. They also appeared to be more spread-out than those cryopreserved with DMSO (FIG. 3B). Moreover, we can observe the presence of cell-to-cell contacts for both the fresh, NA and CA WPEs cryopreserved hepatocytes, whereas no cell-cell contacts were detected for the DMSO cryopreserved hepatocytes (FIG. 3B).

The studies of plating efficiency thus indicate that both DMSO and WPEs cryopreserved hepatocytes performed well with attachment efficiencies in the range of 50% relative to fresh cells. These results were similar to those obtained for DMSO cryopreserved hepatocytes in other studies (22, 23). Furthermore, microscopic analysis of post-thaw hepatocytes seeded on collagen-coated dishes demonstrated their ability to attach and to restore near-normal morphology with cell-to-cell contacts, following cryopreservation with WPE. The attachment properties and cellular morphology were better conserved in hepatocytes that had been cryopreserved with WPE, rather than with DMSO, demonstrating again the higher efficiency of WPEs to cryopreserve rat hepatocytes. The cell-to-cell contacts were present in WPE-cryopreserved rat hepatocytes, suggesting a better conservation of membrane integrity.

### Example 5

### Albumin Secretion by Cryopreserved Rat Hepatocytes

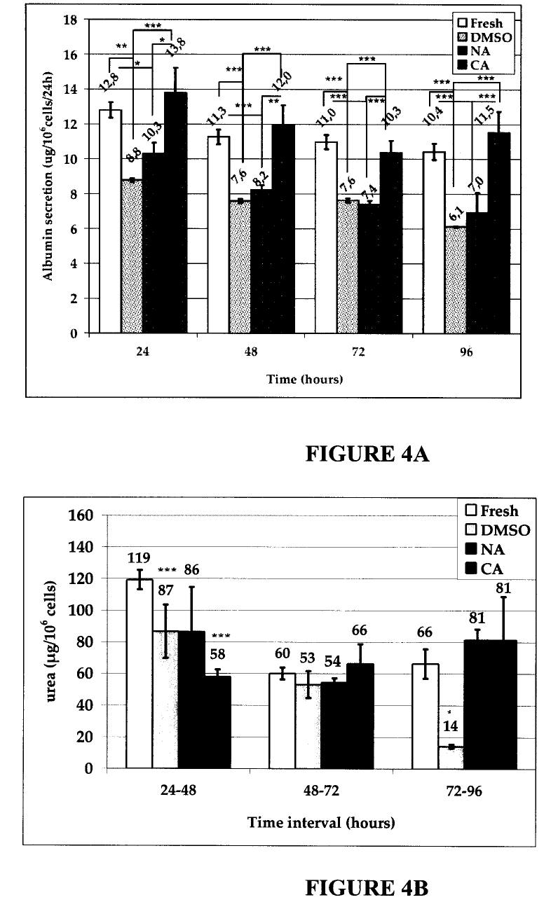

Albumin secretion is a specific marker for protein synthesis in hepatocytes because it requires liver-specific gene expression and intact translational and secretory pathways. The effects of WPEs on albumin production by cryopreserved rat hepatocytes were monitored throughout a 4-day period after plating in culture dishes (FIG. 4A). Albumin secretion by freshly isolated hepatocytes decreased progressively with time from days 1 to 4, although the decrease was much more rapid in cells that had been cryopreserved with DMSO. In fresh hepatocytes, 85% of albumin secretory activity was maintained after 4 days in culture, whereas in DMSO-cryopreserved cells, only 48% of activity remained. However, 83% of albumin secretory activity was maintained in hepatocytes that were cryopreserved with the CA WPEs after 4 days. This value is comparable to that of freshly isolated hepatocytes. When hepatocytes were cryopreserved with NA WPEs, the levels of albumin secretion were approximately 30% less than those of hepatocytes cryopreserved with CA WPEs. These results demonstrate that the hepatospecific function of albumin secretion was well maintained throughout the 4-day culture period in WPE-cryopreserved hepatocytes and that this function was considerably improved with CA WPEs compared to DMSO (FIG. 4A).

### Example 6

### Ammonium Detoxification by Cryopreserved Rat Hepatocytes

The effects of WPEs on ammonium detoxification by cryopreserved rat hepatocytes were measured at days 2, 3 and 4 after plating in culture dishes, compared to fresh cells (FIG. 4B). The production of urea by freshly isolated and DMSO-cryopreserved hepatocytes decreased progressively with time. An important decrease in urea production by the DMSO-cryopreserved hepatocytes was observed on the fourth day after plating, compared to the fresh hepatocytes. Fresh hepatocytes maintained 55% of initial detoxification activity, whereas DMSO-treated cells maintained only 16% of initial activity after 4 days in culture. On the other hand, after subtraction of plant arginase activity, ammonium detoxification in hepatocytes cryopreserved with WPEs was similar to the fresh ones for time intervals of 48-72 and 72-96 h. These findings indicate that the hepatospecific function of ammonium detoxification was well maintained throughout the 4-day culture period with WPE, relative to the DMSO standard (FIG. 4B).

### Example 7

### Cytochrome P450 Enzyme Activities by Cryopreserved Rat Hepatocytes

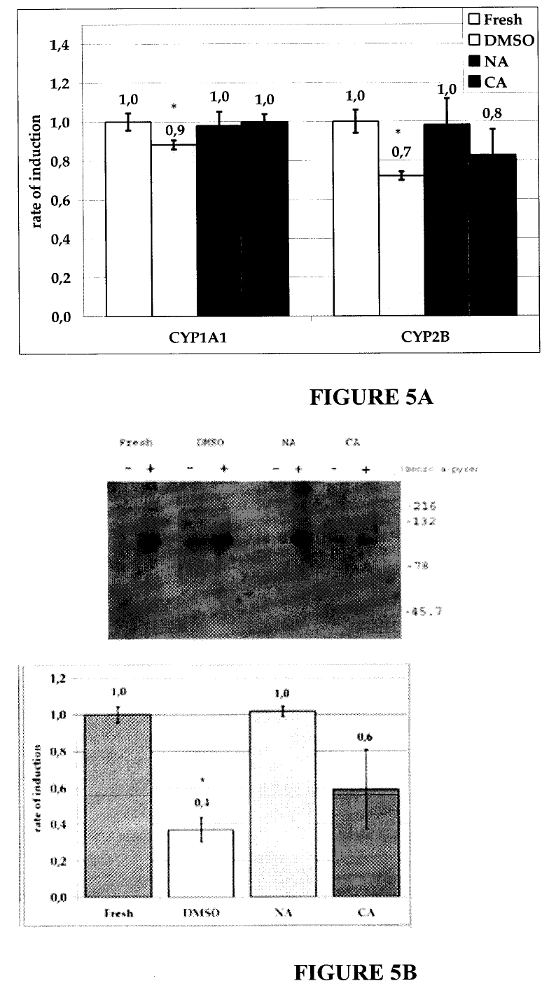

The activity of the xenobiotic-metabolizing cytochrome P450 enzymes was also evaluated as a third marker of hepatospecific functions. Metabolic activity of the cytochrome P450 CYP1A1 and CYP2β isoforms was measured by the EROD (CYP1A1) and PROD (CYP2B) assays after 24 h induction with benzo-a-pyrene (FIG. 5A). Compared to fresh hepatocytes, the relative activity of the P450 CYP1A1 and CYP2B enzymes decreased slightly in DMSO-cryopreserved hepatocytes, while it was maintained in hepatocytes cryopreserved with NA and CA WPEs. Western blot analyses of the CYP1A1 isoform demonstrated that the increase in the benzo-a-pyrene inducible activity was associated with increased expression of the protein (FIG. 5B). This indicates that the metabolic activity of the cytochrome P450 CYP1A1 and CYP2β isoforms was also improved in WPE-cryopreserved hepatocytes, compared to DMSO (FIGS. 5A, B). These results indicate that the WPE-cryopreserved hepatocytes retained their metabolic activity and their capacity to respond to CYP inducers more efficiently than the DMSO cryopreserved rat cells.

### Example 8

### Cryopreservation Potential of the PEs from a Variety of Plant Types on Isolated Rat Hepatocytes

Analysis of the viability of suspensions of rat hepatocytes after freezing was evaluated with the calcein/PI test by flow cytometry, with results shown in FIG. 6. Viability of rat hepatocytes (1.5×10⁶ cells/ml) was evaluated after 7 days of freezing in WME 10% FBS supplemented with 15% DMSO and 50% FBS (DMSO). The effect of PEs from wheat (Triticum aestivum) cv Clair, wheat cv Glenlae, barley (Hordeum vulgare), rye (Secale cereale), alfalfa (Medicago sativa) or spinach (Spinacia oleracea) on viability of the suspension of rat hepatocytes was also evaluated after 7 days of freezing in WME supplemented with 20 mg or 40 mg (+) of NA plant PEs. Freshly isolated hepatocytes (Fresh) served as a reference.

### Example 9

### Cryopreservation of Various Types of Eukaryotic Cells with DMSO and WPEs

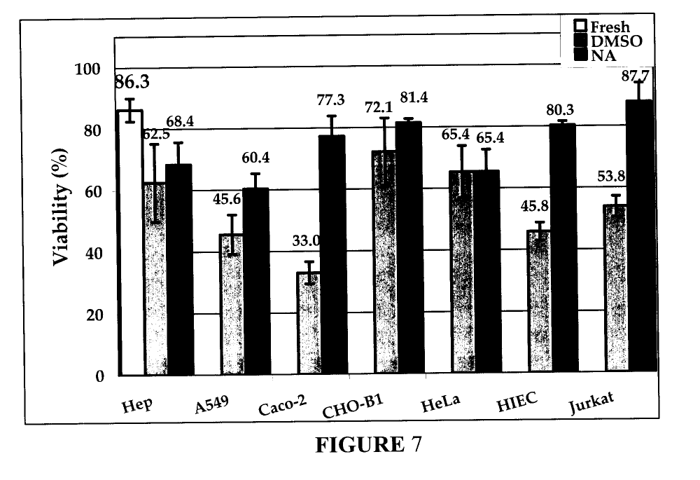

Analysis of the viability of suspensions of eukaryotic cells after freezing was evaluated with calcein/PI test by flow cytometry, with results shown in FIG. 7. Viability of primary rat hepatocytes cells, A549 (human lung carcinoma), Caco-2 (human colorectal adenocarcinoma), CHO-BL (Chinese hamster ovary transfected with TGF-b1 cDNA), HeLa (cervical cancer cells taken from Henrietta Lacks), HIEC (human intestinal epithelium cell) and Jurkat (Human T cell leukemia) cell lines (1.5×10⁶ cells/ml) was evaluated after 7 days of freezing in their respective growth media supplemented with 15% DMSO and 50% FBS (DMSO) or WPEs NA Clair (NA). Freshly isolated hepatocytes (Fresh) served as a reference. These data demonstrates that the WPE is a better cryoprotectant agent than the DMSO.

### Example 10

### Cryopreservation Potential of the WPEs from Ammonium Sulfate Precipitate Fractions on Isolated Rat Hepatocytes

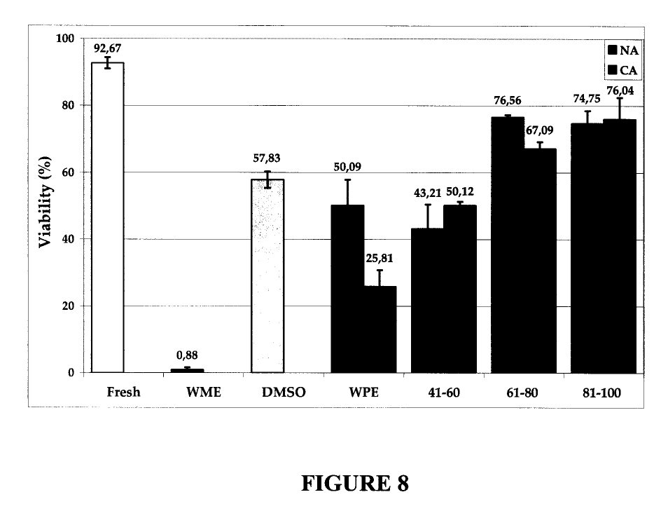

The effect of ammonium sulfate precipitation of the CA extract on cell viability was tested. Ammonium sulfate precipitation is achieved by the addition of ammonium salts to the WPEs to bring up the salt concentration. Proteins start to precipitate as the salt concentration is increased. The collection of separated product is called fractionation; the fraction of the precipitated proteins collected between 41 and 60% of salt saturation is referred to as the 41-60% fraction. Significant results were obtained with the 41-60 fraction of the CA WPE, giving viability of 50.1%, compared to 25.8% for the total CA WPE (FIG. 8). The 61-80 and 81-100% fractions were more efficient giving viabilities were 76.56% and 74.75% respectively with NA extract. For the CA WPE, the viabilities were 67.09 and 76.04%. This demonstrates that the ammonium sulfate precipitation has a positive effect on cell viability. We have also noticed that the protein fractions 41-60, 61-80 and 81-100 were cleaned of their sticky green coloration.

### Example 11

### Influence of the Fetal Bovine Serum on the Cryopreservation Potential of the NA WPE on Isolated Rat Hepatocytes

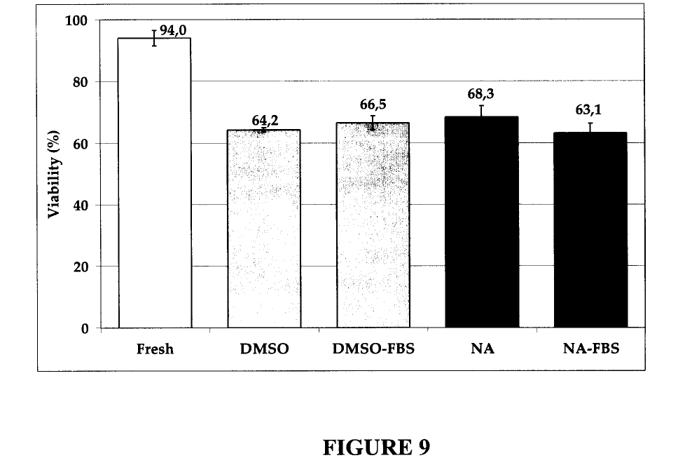

In cryopreservation protocols, fetal bovine serum (FBS) is usually added at concentrations ranging from 10 to 50%. Since FBS is from animal origin, it represents a potential risk of contamination and creates major safety concerns in the industry. Thus, there is increasing interest in developing novel cryopreservation solutions free of products from animal origin. WPE have been tested as a cryopreservant without supplementation of FBS. Non-significant differences in hepatocyte viability were obtained when FBS was added or not to the cryopreservation solutions (FIG. 9). This demonstrates that the addition of bovine serum is not essential for the cryopreservation of primary hepatocytes cells.

### Example 12

### Gluten Content of WPEs

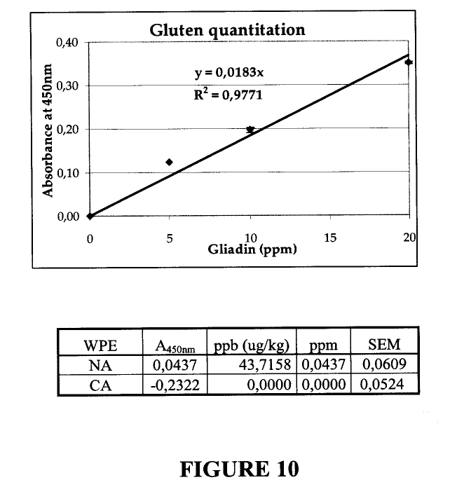

Gluten is a mixture of prolamin and glutelin proteins present in wheat. Coelic disease is a permanent intolerance to gluten that results in damage to the small intestine and is reversible when gluten is avoided by diet. In the Codex Alimentarius “gluten-free” food is defined as food having less than 200 ppm gluten. The proposed new Codex Standard for gluten-free foods defines a maximum content of 20 ppm gluten in naturally gluten-free products and 200 ppm gluten in products rendered gluten-free. Quantitative analyses of the gluten content were obtained for the NA and CA WPEs, giving 0.044 and 0.00 ppm respectively (FIG. 10). This demonstrates that the NA and CA WPEs are gluten-free products.

Abbreviations used herein: freezing tolerance, FT; antifreeze proteins, AFPs; inhibition of ice recrystallization, IRI; wheat protein extracts, WPE; Williams' medium E, WME; dimethyl sulfoxide, DMSO; Leibovitz medium, L-15; 7-ethoxyresorufin-O-deethylase, EROD; 7-pentoxyresorufin-O-depentylase, PROD; propidium iodide, PI; cytochrome P-450, CYP; horseradish peroxidase, HRP; fetal bovine serum, FBS; cold acclimated, CA; non-acclimated, NA; lactate dehydrogenase, LDH; sandwich enzyme-linked immunosorbent assay, ELISA; room temperature, RT; phosphate buffered saline, PBS; PBS supplemented with 0.05% Tween-20, PBS-T; o-phenylendiamine, OPND; tris-buffered saline supplemented with 0.1% Tween-20, TBS-T.

Although the present invention has been described hereinabove by way of specific embodiments thereof, it can be modified, without departing from the spirit and nature of the subject invention as defined in the appended claims.

### REFERENCES

1. Meeks R G, Harrison S D, Bull R J. Hepatotoxicology. CRC Press ed. FL: Boca Raton, 1991.
2. Clement B, Guillouzo A. Cellular and molecular aspects of cirrhosis. John Libbey Eurotext, INSERM ed. Paris: 1992.
3. Glicklis R, Shapiro L, Agbaria R, Merchuk J C, Cohen S. Hepatocyte behavior within three-dimensional porous alginate scaffolds. Biotechnol Bioeng 2000; 67:344-353.
4. Billings R E, McMahon R E, Ashmore J, Wagle S R. The metabolism of drugs in isolated rat hepatocytes. A comparison with in vivo drug metabolism and drug metabolism in subcellular liver fractions. Drug Metab Dispos 1977; 5:518-526.
5. Houston J B. Utility of in vitro drug metabolism data in predicting in vivo metabolic clearance. Biochem Pharmacol 1994; 47: 1469-1479.
6. Kedderis G L. Extrapolation of in vitro enzyme induction data to humans in vivo. Chem Biol Interact 1997; 107:109-121.
7. Zaleski J, Richburg J, Kauffman F C. Preservation of the rate and profile of xenobiotic metabolism in rat hepatocytes stored in liquid nitrogen. Biochem Pharmacol 1993; 46:111.-116.
8. Powis G, Santone K S, Melder D C, Thomas L, Moore D J, Wilke T J. Cryopreservation of rat and dog hepatocytes for studies of xenobiotic metabolism and activation. Drug Metab Dispos 1987; 15:826-832.
9. Santone K S, Melder D C, Powis G. Studies of chemical toxicity to fresh and cryopreserved rat hepatocytes. Toxicol Appl Pharmacol 1989; 97:370-376.
10. Fahy G M. The relevance of cryoprotectant “toxicity” to cryobiology. Cryobiology 1986; 23:1-13.
11. Schneider U, Mazur P. Osmotic consequences of cryoprotectant permeability and its relation to the survival of frozen-thawed embryos. Theriogenology 1984; 21:68-79.
12. Danyluk J, Perron A, Houde M, Limin A, Fowler B, Benhamou N et al. Accumulation of an acidic dehydrin in the vicinity of the plasma membrane during cold acclimation of wheat. Plant Cell 1998; 10:623-638.
13. Seglen P O. Preparation of isolated rat liver cells. Methods Cell Biol 1976; 13:29-83.
14. Guillemette J, Marion M, Denizeau F, Fournier. M, Brousseau P. Characterization of the in vitro hepatocyte model for toxicological evaluation: repeated growth stimulation and glutathione response. Biochem Cell Biol 1993; 71:7-13.
15. Canadian council on animal care. Guide to the care and use of experimental animals. 2nd ed. Canadian council on animal care, 1993.
16. Reader S, Marion M, Denizeau F. Flow cytometric analysis of the effects of tri-n-butyltin chloride on cytosolic free calcium and thiol levels in isolated rainbow trout hepatocytes. Toxicology 1993; 80:117-129.
17. Moldeus P, Hogberg J, Orrenius S. Isolation and use of liver cells. Methods Enzymol 1978; 52:60-71.
18. Moffatt P, Plaa G L, Denizeau F. Rat hepatocytes with elevated metallothionein expression are resistant to N-methyl-N′-nitro-N-nitrosoguanidine cytotoxicity. Toxicol Appl Pharmacol 1996; 136:200-207.
19. Uotila M, Ruoslahti E, Engvall E. Two-site sandwich enzyme immunoassay with monoclonal antibodies to human alpha-fetoprotein. J Immunol Methods 1981; 42:11-15.
20. Wan X, Lachapelle M, Marion M, Fournier M, Denizeau F. Recovery potential of hepatocytes from inhibition of albumin secretion by cadmium. J Toxicol Environ Health 1993; 38:381-392.
21. Laemmli U K. Cleavage of structural proteins during the assembly of the head of bacteriophage T4. Nature 1970; 227:680-685.
22. Silva J M, Day S H, Nicoll-Griffith D A. Induction of cytochrome-P450 in cryopreserved rat and human hepatocytes. Chem Biol Interact 1999; 121:49-63.
23. Sosef M N, Baust J M, Sugimachi K, Fowler A, Tompkins R G, Toner M. Cryopreservation of isolated primary rat hepatocytes: enhanced survival and long-term hepatospecific function. Ann Surg 2005; 241:125-133.

1. A cryopreservation medium comprising a protein-comprising plant extract.

2. The medium of claim 1 wherein said extract is derived from a non-acclimated plant.

3. The medium of claim 1, wherein said extract is derived from the aerial parts or leaf tissue of a plant.

4. The medium of claim 1, wherein said plant is of a plant family selected from Poaceae (Gramineae), Leguminoseae, and Amaranthaceae.

5. The medium of claim 1, wherein said plant is selected from wheat, rye, barley, alfalfa and spinach.

6. The medium of claim 1, wherein said extract is substantially soluble.

7. The medium of claim 1, wherein said extract is protein-enriched.

8-9. (canceled)

10. The medium of claim 1, wherein said medium is substantially free of DMSO.

11. The medium of claim 1, wherein said medium is substantially free of exogenous animal serum.

12. The medium of claim 11, wherein said animal serum is fetal bovine serum.

13. The medium of claim 1, wherein said extract is substantially free of gluten.

14. The medium of claim 1, wherein the viability after thawing of a biological material cryopreserved therein is greater than or equal to 40%.

15-16. (canceled)

17. The medium of claim 1, wherein the level or activity of a functional parameter after thawing of a biological material cryopreserved therein is greater than or equal to 40%.

18-19. (canceled)

20. The medium of claim 17, wherein said functional parameter is selected from plating efficiency, adherence, cellular morphology, cellular secretion, protein synthesis, ammonium detoxification and enzyme activity.

21. The medium of claim 1, wherein said medium is for cryopreservation of a biological material selected from a molecule, organelle, cell, embryo, tissue and organ.

22. The medium of claim 21, wherein said cell is a eukaryotic cell.

23. The medium of claim 21, wherein said cell is a primary cell, a cell line or an immortalized cell.

24. The medium of claim 21, wherein said cell, embryo, tissue or organ is a mammalian cell, embryo, tissue, or organ.

25. The medium of claim 24, wherein said cell, embryo, tissue or organ is a human cell, embryo, tissue or organ.

26. The medium of claim 24, wherein said cell or tissue is a hepatocyte or hepatic tissue.

27. A composition comprising the medium of claim 1 and a biological material.

28-46. (canceled)

47. The composition of claim 27, wherein said biological material is selected from a molecule, organelle, cell, embryo, tissue and organ.

48. The composition of claim 47, wherein said cell is a eukaryotic cell.

49. The composition of claim 47, wherein said cell is a primary cell, a cell line or an immortalized cell.

50. The composition of claim 47, wherein said cell, embryo, tissue or organ is a mammalian cell, embryo, tissue or organ.

51. The composition of claim 50, wherein said cell, embryo, tissue or organ is a human cell, embryo, tissue or organ.

52. The composition of claim 50, wherein said cell or tissue is a hepatocyte cell or hepatic tissue.

53. The composition of claim 27, wherein said composition is frozen.

54. A method for cryopreserving a biological material, said method comprising freezing a suspension of the biological material in the medium of claim 1.

55. A method for cryopreserving a biological material, said method comprising introducing the biological material into the medium of claim 1 and freezing the medium comprising the biological material.

56-74. (canceled)

75. The method of claim 54, wherein said biological material is selected from a molecule, organelle, cell, embryo, tissue, and organ.

76. The method of claim 75, wherein said cell is a eukaryotic cell.

77. The method of claim 76, wherein said cell is a primary cell, a cell line or an immortalized cell.

78. The method of claim 75, wherein said cell, embryo tissue or organ is a mammalian cell, embryo, tissue or organ.

79. The method of claim 78, wherein said cell, embryo, tissue or organ is a human cell, embryo, tissue or organ.

80. The method of claim 75, wherein said cell or tissue is a hepatocyte cell or hepatic tissue.

81. A kit or package comprising the medium of claim 1.

82-108. (canceled)

109. The kit or package of claim 81, further comprising instructions for the cryopreservation of a biological material.

110. (canceled)

111. A method for cryopreserving a biological material, said method comprising introducing a protein-comprising plant extract into a cryopreservation medium prior to freezing.

112-114. (canceled)
